(19) United States
(12) Patent Application Publication
(10) Pub. No.: US 2014/0031619 A1
(43) Pub. Date: Jan. 30, 2014

(54) ERECTION ASSISTANCE DEVICE

(71) Applicant: Il Moon, Seoul (KR)

(72) Inventor: Il Moon, Seoul (KR)

(21) Appl. No.: 13/650,779

(22) Filed: Oct. 12, 2012

(30) Foreign Application Priority Data
Jul. 27, 2012 (KR) 10-2012-0082720
Sep. 7, 2012 (KR) 10-2012-0099301

(51) Int. Cl. A61F 2/26

(52) U.S. Cl. CPC A61F 2/26
USPC 600/40

(57) ABSTRACT

The present invention relates to an erection assistance device that includes: an elastic penis implant which has a semi-cylindrical shape, with its lower pan opened, and is processed to be inserted between an epidermis and a cavernous body of a penis; a storing part which is connected to the expansive part via connection pipes and stores a fluid to be supplied into the expansive part; and a pump which is provided in the middle of the connection pipes, wherein the pump is manipulated to adjust a degree of expansion of the penis implant.

### BACKGROUND OF THE INVENTION

1. Field of the Invention

The present invention relates to an erection assistance device with a novel structure, which is capable of freely controlling an erection state of a penis.

2. Description of the Related Art

In recent years, there have been developed a variety of methods for relief of stress and impotence which result from a small penis.

In particular, as disclosed in Korean Patent Application Publication No. 10-2003-0076028, there has been recently developed a penis implant which is made of soft silicone and is filled with silicone gel or physiological saline.

This penis implant has a semi-cylindrical shape having a longitudinal C-like section when viewed from the front, with its lower part opened, and is processed to be inserted between an epidermis and a cavernous body of the penis.

Such a penis implant has no side effect and provides an advantage of no feeling of irritation as it is not absorbed in a body tissue, not shrunk, not spoiled and not cured after the processing.

However, such a penis implant has a problem of inconvenient daily living as it have a constant thickness to always keep the penis erected.

Thus, there is a need for a novel method to overcome the above problem.

### SUMMARY OF THE INVENTION

Accordingly, it is an object of the present invention to provide an erection assistance device with a novel structure, which is capable of freely controlling an erection state of a penis.

To achieve the above object, according to an aspect of the invention, there is provided an erection assistance device including: an elastic penis implant (10) which has a semi-cylindrical shape, with its lower part opened, and is processed to be inserted between an epidermis (1) and a cavernous body (2) of a penis; a storing part (20) which is connected to the expansive part (13) via connection pipes (31 and 32) and stores a fluid to be supplied into the expansive part (13); and a pump (40) which is provided in the middle of the connection pipes (31 and 32), wherein the pump (40) is manipulated to adjust a degree of expansion of the penis implant (10).

According to one embodiment, the connection pipes (31 and 32) include a supplying pipe (31) and a discharging pipe (32), with their both ends connected to the expansive part (13) and the storing part (20), the pump (40) is provided in the middle of the supplying pipe (31), and the device further includes a valve (50) which is provided in the middle of the discharging pipe (32).

According to another embodiment, the penis implant (10) is made of an elastic silicone material and has a space (11) formed therein to be filled with a cohesive gel (12) used for breast shaping.

According to another embodiment, the pump (40) and the storing part (20) are inserted and installed in a scrotum of a user.

According to another embodiment, the connection pipes (31 and 32) include a supplying pipe (31) and a discharging pipe (32), with their both ends connected to the expansive part (13) and the storing part (20), and the pump (40) includes a supplying pump (43) provided in the middle of the supplying pipe (31) and a discharging pump (44) provided in the middle of the discharging pipe (32).

According to another embodiment, the expansive part (13) is in the form of a tube and is provided in the outer side of the penis implant (10).

According to another embodiment, the penis implant (10) has a plurality of through holes (14) and the expansive part (13) is in the form of a ball and is inserted in and fixed to the inside of the through holes (14).

According to another embodiment, a plurality of fixing portions (15) are formed to project from the circumference of the penis implant (10) and are operable to be fixed to the epidermis (1) of the penis.

According to another embodiment, the connection pipes (31 and 32) are installed to be buried in the surface of the penis implant (10), the expansive part (13) is formed on the surface of the penis implant (10), and a plurality of communicating holes (33) to connect the supplying pipe (31), the discharging pipe (32) and the expansive part (13) are formed in the outer side of the penis implant (10).

The erection assistance device as configured above can freely adjust a degree of expansion of the penis implant (10) by using the pump (40) to supply a fluid into the expansive part (13) of the penis implant (10) or manipulating the valve (50) to discharge the fluid within the expansive part (13) into the storing part (20). Accordingly, a user can freely control a state of erection of his penis, overcoming a problem which may be caused by consistent erection of the penis.

### BRIEF DESCRIPTION OF THE DRAWINGS

The above and/or other aspects and advantages of the present invention will become apparent and more readily appreciated from the following description of the embodiments, taken in conjunction with the accompanying drawings of which:

FIG. 1 is a perspective view showing an erection assistance device in accordance with the present invention.

FIG. 2 is a sectional view showing an erection assistance device in accordance with the present invention.

FIG. 3 is a front sectional view showing a state of procedure of the erection assistance device in accordance with the present invention.

FIG. 4 is a reference view for explaining an operation of the erection assistance device in accordance with the present invention.

FIG. 5 is a reference view showing a modification of the erection assistance device in accordance with the present invention.

FIG. 6 is a perspective view showing a second embodiment of the erection assistance device in accordance with the present invention.

FIG. 7 is a perspective view showing a third embodiment of the erection assistance device in accordance with the present invention.

FIG. 8 is a side sectional view showing the third second embodiment of the erection assistance device in accordance with the present invention.

FIG. 9 is a plan view showing a fourth embodiment of the erection assistance device in accordance with the present invention.

FIG. 10 is a perspective view showing a fifth embodiment of the erection assistance device in accordance with the present invention.

FIG. 11 is a front sectional view showing the fifth embodiment of the erection assistance device in accordance with the present invention.

FIG. 12 is a perspective view showing a modification of the fifth embodiment of the erection assistance device in accordance with the present invention.

### DETAILED DESCRIPTION OF THE PREFERRED EMBODIMENT

The present invention will be now described in detail with reference to the accompanying drawings.

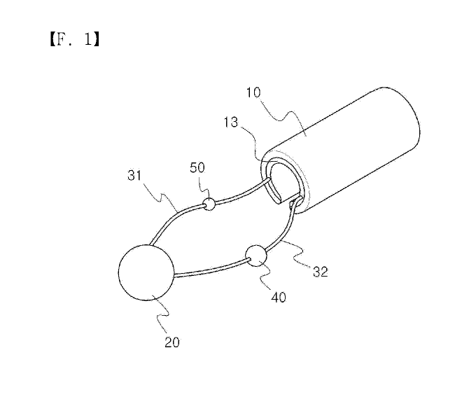

FIGS. 1 to 4 show an erection assistance device in accordance with the present invention. Referring to these figures, an erection assistance device includes an elastic penis implant 10 which is processed to be inserted between an epidermis 1 and a cavernous body 2 of a penis, with an expansive part 13 formed in its one side, a storing part 20 which is connected to the expansive part 13 via connection pipes 31 and 32 and stores a fluid to be supplied into the expansive part 13, and a pump 40 and a valve 50 which are provided in the middle of the connection pipes 31 and 32.

The penis implant 10 has a semi-cylindrical shape with its lower side opened and is made of an elastic silicone material. The penis implant 10 has a space 11 formed therein to be filled with a cohesive gel 12 used for breast shaping.

The expansive part 13 has a semi-cylindrical shape corresponding to the penis implant 10 and is provided in the inner side of the penis implant 10. The expansive part 13 is made of an elastic silicone material and has a space 13a formed therein.

Accordingly, the expansive part maintains an entirely shrunk state at ordinary time by its self-elasticity and is expanded when a fluid is fed into its internal space 13a, such that the penis implant 10 is outward expanded.

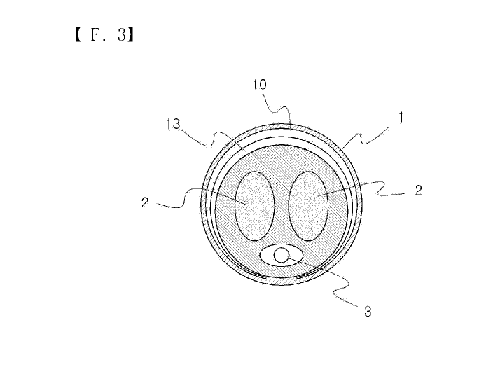

As shown in FIG. 3, the penis implant 10 is configured such that its upper portion is thick and its thickness becomes smaller toward its both sides of an opening of its lower portion. The penis implant 10 is operated to be inserted between the epidermis 1 and the cavernous body 2 of the penis, with the opening directed toward the lower portion, such that the penis implant 10 does not press a penile urethra 3 located below the penis.

The storing part 20 is made of an elastic silicone material and has a shape of airbag having an internal space in which the fluid to be fed into the expansive part is stored. In this example, the fluid may be air or sterilized water.

The connection pipes 31 and 32 connecting the storing part 20 and the expansive part 13 include a supplying pipe 31 and a discharging pipe 32, with their both ends connected to the expansive part 13 and the storing part 20. The pump is provided in the middle of the supplying pipe 31 and the valve 50 is provided in the middle of the discharging pipe 32.

The supplying pipe 31 and the discharging pipe 32 are made of a flexible silicone material.

The pump 40 includes an elastic housing 41 having an inlet hole and an outlet hole formed in its both ends, and check valves 42 formed in the inlet hole and the outlet hole of the housing 41. When the outer side of the housing 41 is pressed, a fluid contained in the housing 41 is supplied into the expansive part 13 via the outlet hole. When the pressing is released, the housing 41 is expanded by its self-elasticity such that the fluid contained in the storing part 20 is introduced into the housing 41 via the inlet hole.

Accordingly, repeatedly pressing the housing 41 of the pump 40 allows the fluid within the storing part 20 to be supplied into the expansive part 13.

The valve 50 is provided in the middle of the discharging pipe 32 and prevents the fluid supplied into the expansive part 13 from being discharged into the storing part 20 via the discharging pipe 32. When a pressing portion formed in one side of the valve 50 is pressed, the discharging pipe 32 is opened to allow the fluid within the expansive part 13 to be discharged into the storing part 20.

As described above, the pump 40 for supplying the fluid into one side of the housing 41 by pressing the housing 41 is a small air pump commonly known in the art and the valve to be opened/closed by pressing its circumference is commonly used in the art, and therefore, explanation of details of which are omitted.

The storing part 20, the pump 40 and the valve 50 are operated and installed such that they are inserted in a scrotum of a user.

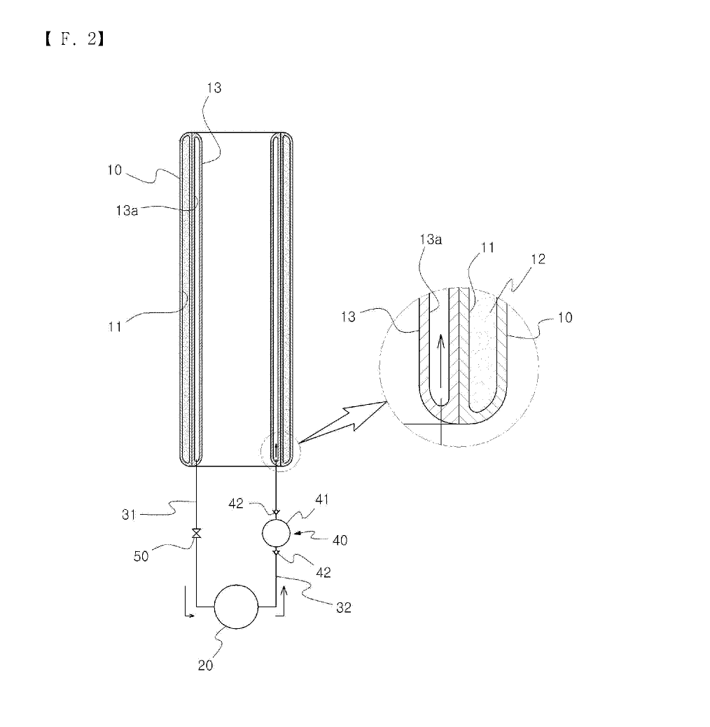

Accordingly, when the user repeatedly presses the pump 40, the fluid within the storing part 40 is supplied into the expansive part 13 of the penis implant 10 such that the penis implant 10 is expanded to erect the penis as shown in FIG. 4. In addition, when the valve 50 is manipulated, the fluid within the expansive part 13 is supplied into the storing part 20 such that the penis implant 10 is shrunk, as shown in FIG. 2, to return the penis to its original size.

The erection assistance device as configured above can freely adjust a degree of expansion of the penis implant 10 by using the pump 40 to supply the fluid into the expansive part 13 of the penis implant 10 or manipulating the valve 50 to discharge the fluid within the expansive part 13 into the storing part 20.

Accordingly, the user can freely control a state of erection of his penis, overcoming a problem which may be caused by consistent erection of the penis.

In addition, since the penis implant 10 is made of an elastic silicone material and has the space 11 formed therein to be filled with the cohesive gel 12 used for breast shaping, it is possible to improve tactile sensation of an outer circumference of the penis implant 10, thereby preventing a pain in sexual intercourse.

In addition, since the pump 40, the storing part 20 and the valve are inserted in the scrotum of the user, it is possible to prevent the pump 40 and the storing part 20 from being externally exposed.

In particular, since the scrotum is formed of a thin skin exposed to the external of the user' body, it is convenient for the user to manipulate the pump 40 and the valve 50.

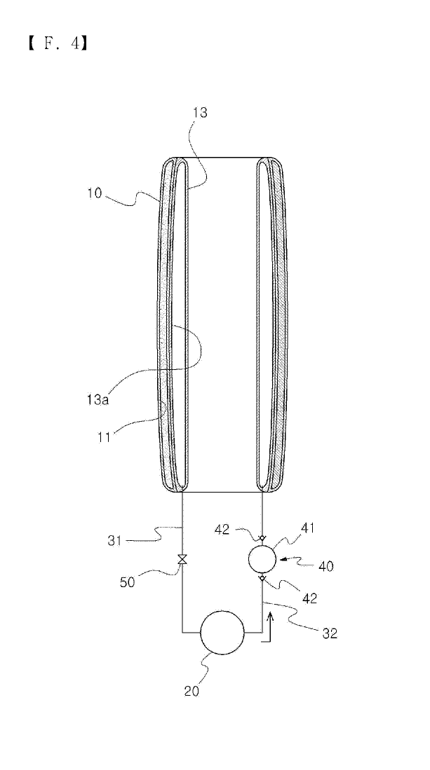

Although it has been illustrated in this embodiment that the pump 40 and the valve 50 are provided in the supplying pipe 31 and the discharging pipe 32, respectively, the valve 50 may be omitted and the pump 40 may be replaced with a supplying pump 43 and a discharging pump 44 which are connected to the supplying pipe 31 and the discharging pump 32, respectively, as shown in FIG. 5. In the configuration shown in FIG. 5, the supplying pump 43 may be manipulated to supply the fluid within the storing part 20 into the expansive part 13 and the discharging pump 44 may be manipulated to discharge the fluid within the expansive part 13 into the storing part 20.

In this configuration, the supplying pump 43 and the discharging pump 33 are connected to the supplying pipe 31 and the discharging part 32 in the opposite direction to each other.

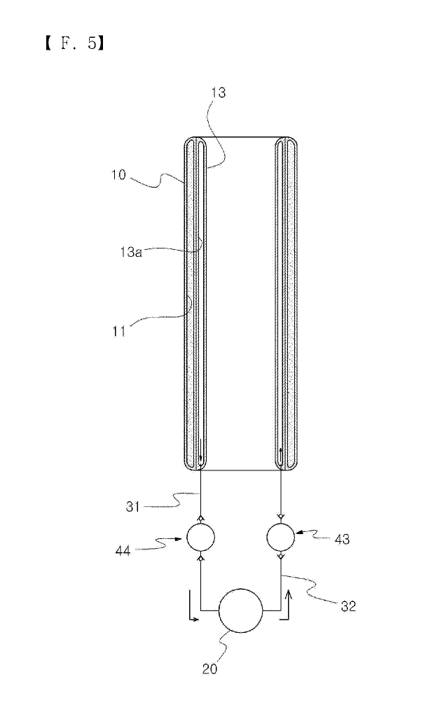

In addition, although it has been illustrated in this embodiment that the expansive part 13 is provided in the inside of the penis implant 10, the expansive part 13 may be in the form of a tube to be fixed to the outer side of the penis implant 10, as shown in FIG. 6.

In this case, when the pump 40 is used to expand the expansive part 13, the expansive part 13 is expanded in the form of a bump at the outer side of the penis implant 10, as indicated by a dashed line in FIG. 6.

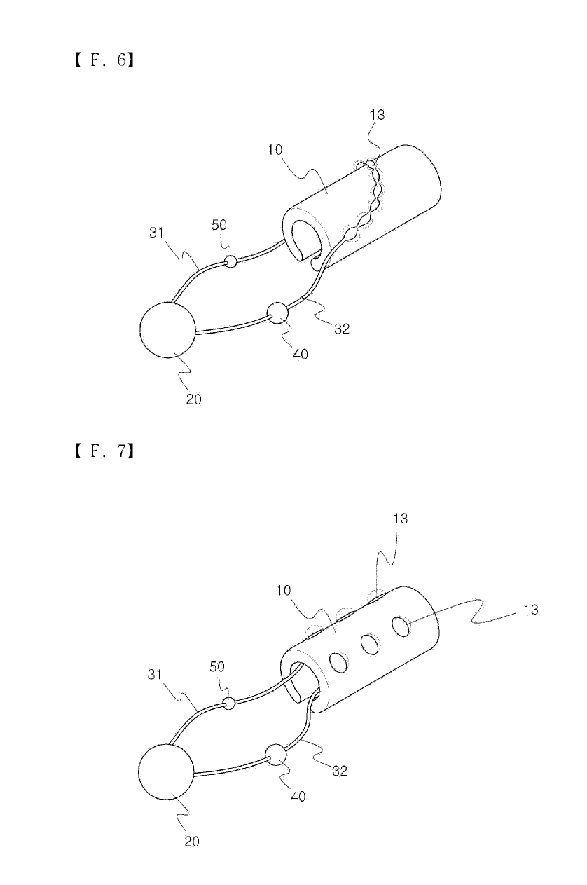

FIGS. 7 and 8 show another embodiment of the present invention. As shown in these figures, the penis implant 10 has a plurality of through holes 14 penetrating through the inner and outer side of the penis implant 10 and the expansive part 13 is in the form of a ball and is inserted in and fixed to the inside of the through holes 14. The supplying pipe 31 and the discharging pipe 32 are provided in the inner side of the penis implant 10 to connect the expansive part 13.

Accordingly, when the pump 40 is used to expand the expansive part 13, the expansive part 13 is expanded in the form of a bump as indicated by a dashed line in FIG. 7.

In this way, the expansive part 13 may be configured in various forms.

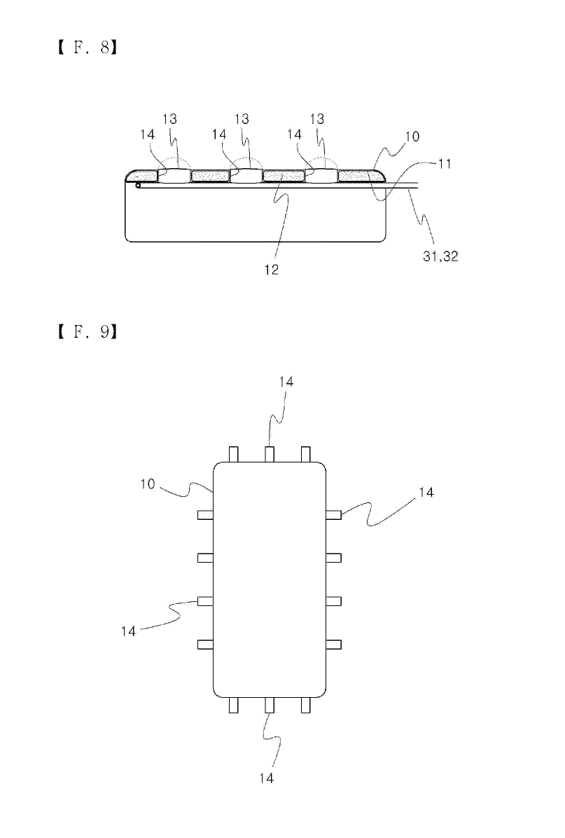

FIG. 9 shows a fourth embodiment of the present invention. As shown, a plurality of fixing portions 15 are formed to project from the circumference of the penis implant 10.

Each of the fixing portions 15 has a long bar or band shape and extends outward from the circumference of the penis implant 10.

Accordingly, by fixing the fixing portions 15 to the epidermis 1 of the penis using a suture thread or the like after inserting the penis implant 10 between the epidermis 1 and the cavernous body 2 of the penis, it is possible to effectively prevent the penis implant 10 from being floated after procedure.

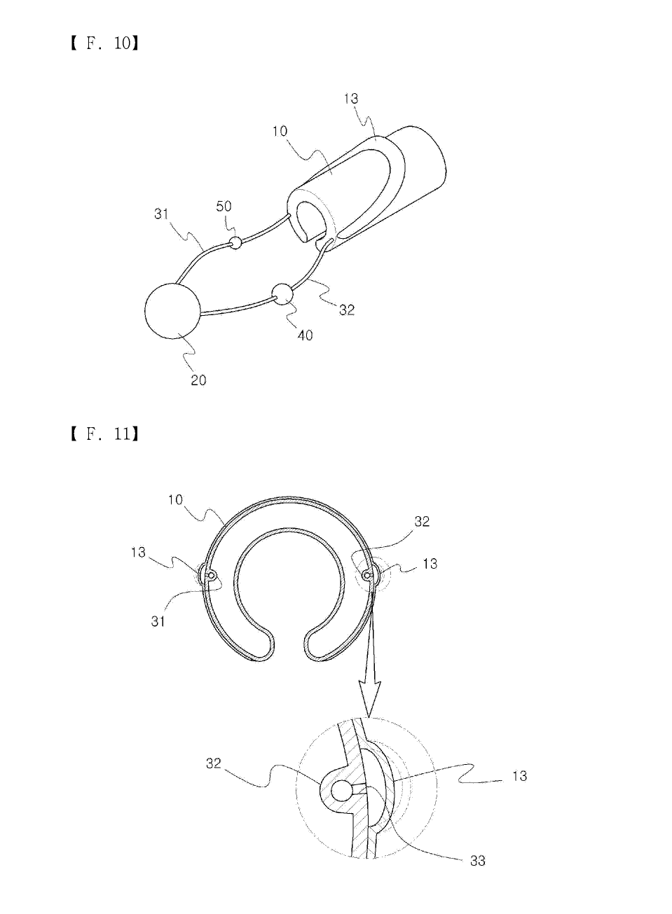

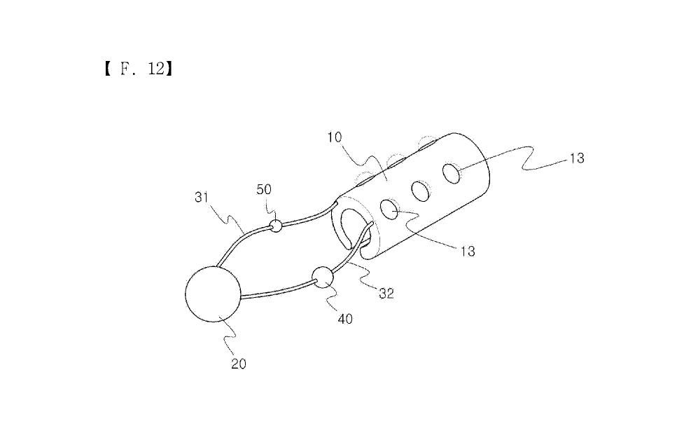

FIGS. 10 and 11 show a fifth embodiment of the present invention. As shown, the supplying pipe 31 and the discharging pipe 32 are installed to be buried in the surface of the penis implant 10 and the expansive part 13 is provided in the form of a tube on the surface of the penis implant 10.

In this case, the expansive part 13 is formed by combining a separate sheet to the surface of the penis implant 10.

In addition, a plurality of communicating holes 33 to connect the supplying pipe 31, the discharging pipe 32 and the expansive part 13 are formed in the outer side of the penis implant 10. A fluid supplied via the supplying pipe 31 is introduced into the expansive part 13 via the communicating holes 33 and a fluid within the expansive part is discharged into the discharging pipe 32 via the communicating holes 33.

FIG. 12 shows a modification of the fifth embodiment. As shown, a plurality of expansive parts 13 are provided in the form of a ball in the outer side of the penis implant 10.

In this manner, the shape of the expansive part 13 and the method of connecting the expansive part 13, the supplying pipe 31 and the discharging pipe 32 may be modified in various ways.

While the present invention has been particularly shown and described with reference to exemplary embodiments thereof, it will be understood by those skilled in the art that various changes in form and details may be made therein without departing from the spirit and scope of the present invention. The exemplary embodiments are provided for the purpose of illustrating the invention, not in a limitative sense. Thus, it is intended that the present invention covers the modifications and variations of this invention provided they come within the scope of the appended claims and their equivalents.

What is claimed is:

1. An erection assistance device comprising:
an elastic penis implant (10) which has a semi-cylindrical shape, with its lower part opened, and is processed to be inserted between an epidermis (1) and a cavernous body (2) of a penis;
a storing part (20) which is connected to the expansive part (13) via connection pipes (31 and 32) and stores a fluid to be supplied into the expansive part (13); and
a pump (40) which is provided in the middle of the connection pipes (31 and 32),
wherein the pump (40) is manipulated to adjust a degree of expansion of the penis implant (10).

2. The erection assistance device according to claim 1, wherein the connection pipes (31 and 32) include a supplying pipe (31) and a discharging pipe (32), with their both ends connected to the expansive part (13) and the storing part (20), and the pump (40) is provided in the middle of the supplying pipe (31),
the device further comprising a valve (50) which is provided in the middle of the discharging pipe (32).

3. The erection assistance device according to claim 1, wherein the penis implant (10) is made of an elastic silicone material and has a space (11) formed therein to be filled with a cohesive gel (12) used for breast shaping.

4. The erection assistance device according to claim 1, wherein the pump (40) and the storing part (20) are inserted and installed in a scrotum of a user.

5. The erection assistance device according to claim 1, wherein the connection pipes (31 and 32) include a supplying pipe (31) and a discharging pipe (32), with their both ends connected to the expansive part (13) and the storing part (20), and the pump (40) includes a supplying pump (43) provided in the middle of the supplying pipe (31) and a discharging pump (44) provided in the middle of the discharging pipe (32).

6. The erection assistance device according to claim 1, wherein the expansive part (13) is in the form of a tube and is provided in the outer side of the penis implant (10).

7. The erection assistance device according to claim 1, wherein the penis implant (10) has a plurality of through holes (14) and the expansive part (13) is in the form of a ball and is inserted in and fixed to the inside of the through holes (14).

8. The erection assistance device according to claim 1, wherein a plurality of fixing portions (15) are formed to project from the circumference of the penis implant (10) and are operable to be fixed to the epidermis (1) of the penis.

9. The erection assistance device according to claim 1, wherein the connection pipes (31 and 32) are installed to be buried in the surface of the penis implant (10), the expansive part (13) is formed on the surface of the penis implant (10), and a plurality of communicating holes (33) to connect the supplying pipe (31), the discharging pipe (32) and the expansive part (13) are formed in the outer side of the penis implant (10).
